SEQUENCE LISTING (20 sequences)

SEQ ID NO: 1 - protein, 89 aa, Artificial
  SDF-1 Alpha
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 2 - protein, 93 aa, Artificial
  SDF-1 Beta
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 3 - protein, 119 aa, Artificial
  SDF-1 Gamma
MNAKVVVVLVLVLTALCLSDGKPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 4 - protein, 140 aa, Artificial
  SDF-1 Delta
MNAKVVVVLVLVLTALCLSDGKPVSLSYRCPCRFFESHVARANVKHLKILNTPNCALQIVARLKNNNRQVCIDPKLKWIQEYLEKALNNLISAAPAGKRVIAGARALHPSPPRACPTARALCEIRLWPPPEWSWPSPGDV

SEQ ID NO: 5 - protein, 90 aa, Artificial
  SDF-1 Epsilon
MNAKVVVVLVLVLTALCLSDGKPVSLSYRCPCRFFESHVARANVKHLKILNTPNCALQIVARLKNNNRQVCIDPKLKWIQEYLEKALNNC

SEQ ID NO: 6 - protein, 100 aa, Artificial
  SDF-1 Phi
MNAKVVVVLVLVLTALCLSDGKPVSLSYRCPCRFFESHVARANVKHLKILNTPNCALQIVARLKNNNRQVCIDPKLKWIQEYLEKALNKIWLYGNAETSR

SEQ ID NO: 7 - DNA, 20 bp, Artificial
  Primer
tgcaccaccaactgcttagc

SEQ ID NO: 8 - DNA, 21 bp, Artificial
  Primer
ggcatggactgtggtcatgag

SEQ ID NO: 9 - DNA, 24 bp, Artificial
  Primer
aagcaaacccaaatctcacctggc

SEQ ID NO: 10 - DNA, 24 bp, Artificial
  Primer
tcaccatcaagcctgctccattct

SEQ ID NO: 11 - DNA, 20 bp, Artificial
  Primer
tcgcctggcaccgtaggaca

SEQ ID NO: 12 - DNA, 20 bp, Artificial
  Primer
ggatgcggaatgcccgtcgt

SEQ ID NO: 13 - DNA, 20 bp, Artificial
  Primer
caactggcaggactttctca

SEQ ID NO: 14 - DNA, 20 bp, Artificial
  Primer
ttctttgatgcctgtcttcg

SEQ ID NO: 15 - DNA, 20 bp, Artificial
  Primer
ttgccaacgatcgtggtcat

SEQ ID NO: 16 - DNA, 20 bp, Artificial
  Primer
ggatcatggggcttgcctaa

SEQ ID NO: 17 - DNA, 21 bp, Artificial
  Primer
accaaagctcacgcgtggaaa

SEQ ID NO: 18 - DNA, 22 bp, Artificial
  Primer
tgatgtgtctctcggtcaagtt

SEQ ID NO: 19 - DNA, 21 bp, Artificial
  Primer
tggaccaagggcttcaaggcc

SEQ ID NO: 20 - DNA, 21 bp, Artificial
  Primer
catgtagcaggcattgcagcc

(19) United States
(12) Patent Application Publication
(10) Pub. No.: US 2020/0323784 A1
(43) Pub. Date: Oct. 15, 2020

(54) USE OF CXCL12 TO PROMOTE SURVIVAL, FUNCTION, AND IMMUNOISOLATION OF STEM CELL-DERIVED BETA CELLS

(71) Applicant: The General Hospital Corporation, Boston, MA (US)

(72) Inventors: Mark Poznansky, Newton, MA (US); David Alagpulinsa, Boston, MA (US); Timothy Brauns, Brookline, MA (US)

(21) Appl. No.: 16/648,569

(22) PCT Filed: Sep. 20, 2018
(86) PCT No.: PCT/US18/51950

§ 371 Date: Mar. 18, 2020

Related U.S. Application Data
(60) Provisional application No. 62/561,058, filed on Sep. 20, 2017.

(51) Int. Cl. A61K 9/50; C07K 14/52; C12N 5/0781; A61K 35/17; A61P 3/10

(52) U.S. Cl. CPC A61K 9/50; C07K 14/521; A61K 38/00; A61K 35/17; A61P 3/10; C12N 5/0635

(57) ABSTRACT

The present invention relates to compositions comprising at least one in vitro-developed β-cell and a CXCL12 polypeptide encapsulated in a microcapsule. The invention further relates to methods for treating diabetes, accelerating the normalization of hyperglycemia, and preventing fibrotic pericapsular overgrowth of microcapsules using transplanted human stem cell-derived β cells co-encapsulated with a CXCL12 polypeptide.

### RELATED APPLICATIONS

This application claims the benefit, under 35 U.S.C. § 119(e), of U.S. Provisional Application No. 62/561,058, filed Sep. 20, 2017, the entire contents of which are incorporated by reference herein.

### FIELD OF THE INVENTION

The present invention relates to compositions and methods for treating hyperglycemia, and more particularly, using transplanted human stem cell-derived (3 cells via co-encapsulation with a CXCL12 polypeptide.

### BACKGROUND OF THE INVENTION

Type 1 diabetes (T1D) is characterized by autoimmune-mediated destruction of the insulin-producing pancreatic β-cells. It is one of the oldest known diseases that afflict man and remains incurable to date. Exogenous insulin administration, which is the current treatment of choice for T1D does not mimic the dynamic β-cell responses to glucose fluctuations and patients ultimately succumb to complications. An ideal cure for T1D would be to replace the lost cells with identically functional β-cells. Replacement therapy using human cadaveric islets has demonstrated proof-of-principle to reverse diabetes. However, the extreme scarcity of healthy donor islets and the requirement for lifelong systemic immunosuppression limit the practicality of this treatment modality.

The recent breakthroughs in generating functional β-cells from human pluripotent stem cells in vitro, the so-called SC-β cells, makes the use of β-cell replacement to cure T1D a close reality, given the feasibility of these protocols to generate scalable quantities of SC-β cells. However, potential immune rejection, limited survival and function and potential teratoma formation by potential undifferentiated cells are limiting factors constraining their clinical application. Even autologous SC-β cells would be immunogenic in the setting of autoimmune T1D since prevailing β-cell autoreactive immune cells would attack the transplanted β-cells.

Furthermore, manipulations with differentiation molecules could cause altered expression of potential antigens that elicit immune responses following transplantation. Encapsulation of pancreatic islet n-cells, especially with alginate biomaterial is widely explored as a viable modality for islet replacement therapy. This strategy provides isolation of the islet cells from direct contact with the recipient's immune cells and large molecular weight immunoglobulins, while allowing diffusion of hormones, oxygen, nutrients and waste products. Encapsulation also serves to contain potentially undifferentiated cells and/or teratomas. However, even empty clinical grade alginate microcapsules elicit immune responses, and islet-containing alginate microcapsules elicit foreign body responses characterized by cellularization of the capsule and compromised survival and function of the islets. Moreover, low molecular weight pro-inflammatory cytokines such as IL-1β, TNF-α and CCL2 (MCP-1), secreted by either the encapsulated islet cells or the host cells, can diffuse through the alginate microcapsules and elicit inflammatory cellular responses, pericapsular cellular overgrowth and toxicity to the encapsulated islet cells. Strategies that enhance immune tolerance, long-term survival, and function of SC-β cells are therefore important for their clinical application, irrespective of whether they are autologous or allogeneic.

### SUMMARY OF THE INVENTION

The present invention is based, in part, on the development of compositions and methods for enhancing immune tolerance, long-term survival, and function of SC-β cells and the use thereof to treat diabetes.

Accordingly, one aspect of the invention relates to composition comprising at least one in vitro-developed β-cell and a CXCL12 polypeptide encapsulated in a microcapsule.

Another aspect of the invention relates to a method of treating diabetes in a subject in need thereof, comprising delivering to the subject an effective amount of the composition of the invention, thereby treating diabetes.

A further aspect of the invention relates to a method of accelerating the normalization of hyperglycemia in a subject in need thereof, comprising delivering to the subject an effective amount of the composition of the invention, thereby accelerating the normalization of hyperglycemia.

An additional aspect of the invention relates to a method of preventing fibrotic pericapsular overgrowth of microcapsules in a subject, comprising delivering to the subject an effective amount of the composition of the invention, thereby preventing fibrotic pericapsular overgrowth of the microcapsules.

Another aspect of the invention relates to method for enhancing a response against diabetes in a subject in need thereof, comprising delivering to the subject an effective amount of the composition of the invention, thereby enhancing the response against diabetes.

These and other aspects of the invention are set forth in more detail in the description of the invention below.

### DESCRIPTION OF THE FIGURES

FIGS. 1A-1D show characterization and alginate encapsulation SC-β cell clusters. Representative phase contrast microscopic images (4× magnification) of naked SC-β cell clusters (A) and alginate-encapsulated SC-β cell clusters (B). (C) Representation of the insulin C-peptide positive cells in SC-β cell clusters. SC-β clusters were dispersed into single cell suspensions and subjected to intracellular staining for the indicated antigens and analyzed by flow cytometry as described in methods and materials. (D) Confocal microscopy of microcapsules.

FIGS. 2A-2C show CXCL12 enhances the glucose-stimulated insulin secretion (GSIS) of SC-β cells. (A) Effect of CXCL12 on GSIS of naked SC-β cell clusters. The SC-β cell clusters were pretreated with the indicated concentrations of CXCL12 for 24 h and subjected to basal (2 mM) and high (20 mM) glucose stimulations. The amounts of human C-peptide secreted in the supernatant were determined with a human C-peptide ELISA kit and the total protein of the lysed cells determined with a BCA test kit. The amount of secreted C-peptide was normalized to the total protein content of the cells. (B) Effect of CXCL12 the GSIS index of SC-β cell clusters. The amount of C-peptide secreted under high glucose stimulation was divided by that under basal stimulation as described in (A) to obtain the GSIS index. (C) Effect of alginate co-encapsulation of SC-β cell clusters with CXCL12. After encapsulating SC-β cell clusters into alginate microcapsules and overnight 24 h culture in culture medium, the microencapsulated cells were subjected to GSIS as described for the naked clusters and the GSIS indices calculated as described in (B).

FIGS. 3A-3B show the effect of CXCL12 on cytokine-induced apoptosis of SC-β cell clusters and expression of β cell survival and function genes. (A) SC-β cell clusters were treated with the indicated concentrations of CXCL12 for 24 h and cDNA synthesized from isolated total RNA used for RT-qPCR with primers to the indicated genes and Gapdh used as internal control gene. The expression of each gene mRNA was normalized to that of non-treated cells. (B) Anti-apoptotic effect of CXCL12 on SC-β cell clusters. SC-β cell clusters were seeded into 12-well plates and pre-treated with varying doses of CXCL12 followed by 48 h incubation with a cocktail of cytokines comprising 0.05 μg/mL IL-1β, 0.25 μg/mL TNF-α and 1.8 μg/mL INF-γ. Cell extracts were subjected to active caspase 3 ELISA and the caspase 3 activity normalized to the total protein content determined using a BCA test. Results represent mean±SEM of two biological replicates.

FIGS. 4A-4B show co-encapsulation of SC-β cell clusters with CXCL12 provides enhanced immunoisolation in vivo. (A-B) STZ-induced diabetic mice were implanted with the alginate microcapsules containing the equivalent of 300 SC-β clusters and blood glucose levels monitored up to 12 weeks. Microcapsules were then retrieved from mice and subjected to phase contrast microscopy, H&E staining and immunofluorescent (IF) staining for immune responses. (A, top panel): Phase contrast microscopic images, (A, middle panel): H&E stain and (A, bottom panel): IF images of indicated markers, of microcapsules without CXCL12 retrieved from diabetic mice 12 weeks post-transplantation. (B, top panel): Phase contrast microscopic images, (B, middle panel): H&E stain and (B, bottom panel): IF images of indicated markers, of microcapsules containing 2.0 μg/ml CXCL12 retrieved from diabetic mice 12 weeks post-transplantation.

FIGS. 5A-5I show long-term glycemic control using co-encapsulated SC-β cell clusters and CXCL12 in sensitized mice. STZ-induced diabetic C57BL/6 mice were sensitized to SC-β cells and implanted with 400 equivalent SC-0 cell clusters in alginate microcapsules. Blood glucose levels were monitored and mice were considered hyperglycemic if blood glucose readings are ≥250 mg/dl on two consecutive occasions and hence considered non-surviving. (A) Average plasma glucose readings for diabetic mice treated as indicated. (B). Intraperitoneal glucose tolerance test (IPGTT). Three weeks after transplantation, mice were fasted for 6 h with access to water and injected intraperitoneally with 2 g/kg body weight of glucose and the blood glucose levels measured at the indicated time points. Results represent the mean±SEM. (C) Serum human insulin C-peptide levels in mice implanted as indicated in (A) was determined on serum from blood drawn via retro-orbital bleed on weeks 6 (blue bars) and 18 (red bars) post-transplantation using a commercially available human C-peptide ELISA kit. Results represent the mean±SEM. (D) Intraperitoneal glucose tolerance test (IPGTT). 150 days after transplantation, mice were fasted for 6 h with access to water and injected intraperitoneally with 2 g/kg body weight of glucose and the blood glucose levels measured at the indicated time points. Results represent the mean±SEM. (E) Kaplan-Meier survival curve for mice implanted with indicated treatments. A plasma glucose reading below 250 mg/dl is considered survival while plasma glucose readings above 250 mg/dl on two consecutive occasions is regarded death. (F) Average plasma glucose levels of mice treated as indicated at days 0, 50, 100 and 150. (G) Phase contrast microscopic images (top panel) and H&E stain (bottom panel) on microcapsules without CXCL12 retrieved from mice described in (A) 150 days post-implantation. (H) Phase contrast microscopic images (top panel) and H&E stain (bottom panel) on microcapsules with 0.2 μg/ml CXCL12 retrieved from mice described in A 150 days post-implantation. (I) Phase contrast microscopic images (top panel) and H&E stain (bottom panel) on microcapsules with 2.0 μg/ml CXCL12 retrieved from mice described in (A) 150 days post-implantation.

FIGS. 6A-6B show the function of SC-β cells in alginate microcapsules in immune competent mice. (A) Glycemic control by SC-β cell clusters in alginate microcapsules. STZ-induced diabetic mice were implanted with the alginate microcapsules containing the equivalent of 300 SC-β clusters and plasma glucose levels monitored. (B) Whole blood was drawn from mice implanted with SC-β clusters in alginate microcapsules described in (A) via retro-orbital bleed at weeks 3 (blue bars) and 6 (red bars) post-transplant and serum separated. The serum was subjected to human insulin C-peptide ELISA. Results represent the mean±SEM (n=5).

FIGS. 7A-7C show the function of SC-β cells in alginate microcapsules. STZ-induced diabetic C57BL/6 mice were sensitized to SC-β cells and implanted with 400 equivalent SC-β cell clusters in alginate microcapsules. (A) Intraperitoneal glucose tolerance test (IPGTT). Three weeks after transplantation, mice were fasted for 6 h with access to water and injected intraperitoneally with 2 g/kg body weight of glucose and the blood glucose levels measured at the indicated time points described. The area under the curve (AUC) for glucose over this time course was calculated. Results represent the mean±SEM. (B) Intraperitoneal glucose tolerance test (IPGTT) 150 days post-transplant. IPGTT was carried out as in (A) and AUC for glucose calculated. Results represent the mean±SEM. One-way ANOVA was used to determine significance of differences of means across groups with Bonferroni post hoc test. Results represent the mean±SEM. (C) Mouse C-peptide levels. Mice were retro-orbital bled on day 150 post-implantation with healthy and diabetic non-treated mice used as controls. The serum levels of mouse C-peptide were determined using an ELISA kit that detects mouse C-peptide and not human C-peptide. Results represent the mean±SEM.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. In case of conflict, the present application, including definitions will control. All publications, patent applications, patents, patent publications and other references cited herein are incorporated by reference in their entireties for the teachings relevant to the sentence and/or paragraph in which the reference is presented.

By “SC-β cells” is meant functional β-cells developed from inducible pluripotent stem cells (iPSCs) in vitro.

By “CXCL12 or SDF-1 polypeptide” is meant a protein or fragment thereof that binds a CXCL12 specific antibody and that has chemotaxis or chemorepellant activity. Chemotaxis or chemorepellant activity is determined by assaying the direction of T cell migration (e.g., toward or away from an agent of interest). See, for example, Poznansky et al., Nature Medicine 2000, 6:543-8.

A “subject” is a vertebrate, including any member of the class mammalia, including humans, domestic and farm animals, and zoo, sports or pet animals, such as mouse, rabbit, pig, sheep, goat, cattle and higher primates.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

In this disclosure, “comprises,” “comprising,” “containing” and “having” and the like have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### Compositions and Methods of the Invention

Compositions of the invention are directed to at least one cell and a CXCL12 polypeptide encapsulated in an microcapsule.

CXCL12 polypeptides are known in the art. See, for example, Poznansky et al., Nature Medicine 2000, 6:543-8. Note that the terms CXCL12 and SDF-1 may be used interchangeably. In one embodiment, a CXCL12 polypeptide has at least about 85%, 90%, 95%, or 100% amino acid sequence identity to NP.001029058 and has chemokine or chemorepellant activity. Exemplary SDF1 Isoforms are provided in Table 1.

TABLE 1
HUMAN SDF1 ISOFORMS

| | | Accession | | SEQ |
|---|---|---|---|---|
| | Accession | Number | | ID |
| Name | Number | Versions | Sequence | NO. |
| SDF-1 | NP_954637 | NP_954637.1 | MNAKVVVVLV | SEQ |
| Alpha | | GI:40316924 | LVLTALCLSD | ID |
| | | | GKPVSLSYRC | NO: |
| | | | PCRFFESHVA | 1 |
| | | | RANVKHLKIL | |
| | | | NPTNCALQIV | |
| | | | ARLKNNNRQV | |
| | | | CIDPKLKWIQ | |
| | | | EYLEKALNK | |
| SDF-1 | P48061 | P48061.1 | MNAKVVVVLV | SEQ |
| Beta | | GI:1352728 | LVLTALCLSD | ID |
| | | | GKPVSLSYRC | NO: |
| | | | PCRFFESHVA | 2 |
| | | | RANVKHLKIL | |
| | | | NPTNCALQIV | |
| | | | ARLKNNNRQV | |
| | | | CIDPKLKWIQ | |
| | | | EYLEKALNKR | |
| | | | FKM | |
| SDF-1 | NP_ | NP_001029058.1 | MNAKVVVVLV | SEQ |
| Gamma | 001029058 | GI:76563933 | LVLTALCLSD | ID |
| | | | GKPVSLSYRC | NO: |
| | | | PCRFFESHVA | 3 |
| | | | RANVKHLKIL | |
| | | | NPTNCALQIV | |
| | | | ARLKNNNRQV | |
| | | | CIDPKLKWIQ | |
| | | | EYLEKALNKG | |
| | | | RREEKVGKKE | |
| | | | KIGKKKRQKK | |
| | | | RKAAQKRKN | |
| SDF-1 | | Yu et al. | MNAKVVVVLV | SEQ |
| Delta | | Identification | LVLTALCLSD | ID |
| | | and expression | GKPVSLSYRC | NO: |
| | | of novel | PCRFFESHVA | 4 |
| | | isoforms of | RANVKHLKIL | |
| | | human stromal | NTPNCALQIV | |
| | | cell-derived | ARLKNNNRQV | |
| | | factor 1. Gene | CIDPKLKWIQ | |
| | | (2006) vol. 374 | EYLEKALNNL | |
| | | pp. 174-9 | ISAAPAGKRV | |
| | | | IAGARALHPS | |
| | | | PPRACPTARA | |
| | | | LCEIRLWPPP | |
| | | | EWSWPSPGDV | |
| SDF-1 | | Yu et al. | MNAKVVVVLV | SEQ |
| Epsilon | | Identification | LVLTALCLSD | ID |
| | | and expression | GKPVSLSYRC | NO: |
| | | of novel | PCRFFESHVA | 5 |
| | | isoforms of | RANVKHLKIL | |
| | | human stromal | NTPNCALQIV | |
| | | cell-derived | ARLKNNNRQV | |
| | | factor 1. Gene | CIDPKLKWIQ | |
| | | (2006) vol. 374 | EYLEKALNNC | |
| | | pp. 174-9 | | |
| SDF-1 | | Yu et al. | MNAKVVVVLV | SEQ |
| Phi | | Identification | LVLTALCLSD | ID |
| | | and expression | GKPVSLSYRC | NO: |
| | | of novel | PCRFFESHVA | 6 |
| | | isoforms of | RANVKHLKIL | |
| | | human stromal | NTPNCALQIV | |
| | | cell-derived | ARLKNNNRQV | |
| | | factor 1. Gene | CIDPKLKWIQ | |
| | | (2006) vol. 374 | EYLEKALNKI | |
| | | pp. 174-9 | WLYGNAETSR | |

In another embodiment, the sequence of an exemplary CXCL12/SDF-1 polypeptide is

(SEQ ID NO: 3)
MNAKVVVVLVLVLTALCLSDGKPVSLSYRCPCRFFESHVARANVKHL
KILNTPNCALQIVARLKNNNRQVCIDPKLKWIQEYLEKALNKG
RREEKVGKKEKIGKKKRQKKRKAAQKRKN.

In yet another embodiment, a CXCL12 polypeptide has at least about 85%, 90%, 95%, or 100% amino acid sequence identity to a CXCL12 isoform delta polypeptide and has chemokine or chemorepellant activity. The sequence of an exemplary CXCL12 isoform delta polypeptide is

(SEQ ID NO: 4)
MNAKVVVVLVLVLTALCLSDGKPVSLSYRCPCRFFESHVARANVKHL
KILNTPNCALQIVARLKNNNRQVCIDPKLKWIQEYLEKALNNLISAAP
AGKRVIAGARALHPSPPRACPTARALCEIRLWPPP EWSWPSPGDV.

In some embodiments, a CXCL12 polypeptide is an active fragment or a modified polypeptide that substantially retains at least one biological activity of CXCL12, e.g., chemorepellant activity. “Substantially retains,” as used herein, refers to a level of biological activity that is at least 50% of the biological activity of wild-type CXCL12. Examples of modified CXCL12 polypeptides are disclosed in Application No. 62/454,428, the contents of which are incorporated herein by reference.

CXCL12 polypeptide eluting matrices are characterized, for example, by a release of the CXCL12 polypeptide at a rate of at least about 1.0 ng/mL/hr, e.g., between about 1.0 ng/mL/hr to about 3 ng/mL/hr. In specific embodiments, the CXCL12 polypeptide is released at a rate of about 1.75 ng/ml/hr. The CXCL12 polypeptide is present in the matrix at a concentration of at least about 100 ng/mL, e.g., between about 100 ng/ml to about 1 μg/ml. In certain embodiments, the CXCL12 polypeptide is present in the matrix at a concentration of between about 0.2 μg/ml to about 2.0 μg/ml. In specific embodiments, the CXCL12 polypeptide is present in the matrix at a concentration of between about 100 ng/ml to about 1 μg/ml for about 3 months to about 2 years. Concentrations, release rates and durations will vary according to the selected cell type and disorder to be treated and the selection of appropriate parameters will be known or apparent to those skilled in the art. In general, the CXCL12 polypeptide is released at a rate sufficient to repel effector T-cells from a specific anatomic site. The ability of a CXCL12 polypeptide eluting matrix to repel effector T-cells can be assessed in vitro, using a Boyden chamber assay, as previously described in Poznansky et al., Journal of clinical investigation, 109, 1101 (2002).

Eluting matrices can comprise biocompatible polymers known in the art that are inert to encapsulated cells (i.e., no stimulation or inhibition of cell signaling), and are permeable to the CXCL12 polypeptide to be eluted and the molecule to be sensed (e.g., glucose). The matrix thickness is about 200 about 500 microns and in specific embodiments, forms a capsule around the cells. The diameter of the capsules may be in the range of about 400 μm to about 1000 μm, e.g., about 600 μm to about 700 μm. Biocompatible polymers can be biodegradable or non-degradable. The biocompatible polymer can be carbohydrate-based, protein-based, and/or synthetic, e.g., PLA. Biocompatable materials suitable for use in matrices include, but are not limited to, poly-dimethyl-siloxane (PDMS), poly-glycerol-sebacate (PGS), polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, non-degradable polyurethanes, polystyrenes, polyvinyl chloride, polyvinyl fluoride, polyvinyl imidazole, chlorosulphonated polyolefins, polyethylene oxide, polyvinyl alcohol, nylon silicon, poly(styrene-block-butadiene), polynorbornene, and hydrogels. Other suitable polymers can be obtained by reference to The Polymer Handbook, 3rd edition (Wiley, N.Y., 1989). Combinations of these polymers may also be used.

In one embodiment, CXCL12 polypeptide eluting matrices of the invention further comprise a secondary layer of cells that express the CXCL12 polypeptide, such as mesothelial cells. In other embodiments, the outer layer of the matrix further comprises an absorbable layer of a CXCL12 polypeptide.

In one embodiment, the eluting matrix comprises an alginate (e.g., alginic acid) and generally refers to a carbohydrate polymer (e.g., a polysaccharide) comprising at least two uronate sugars. The uronate sugars can include, but are not limited to, salts of mannuronic acid (or mannuronate), salts of guluronic acid (or guluronate), and/or isomers thereof. In some embodiments, the alginate can be a linear carbohydrate polymer (e.g., a polysaccharide) comprising mannuronate, guluronate and/or isomers thereof. In some embodiments, alginate can be a co-carbohydrate polymer of mannuronate, guluronate, and/or isomers thereof.

As used herein, the term “isomers” refers to compounds having the same molecular formula but differing in structure. Isomers which differ only in configuration and/or conformation are referred to as “stereoisomers.” The term “isomer” is also used to refer to an enantiomer. The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate plane-polarized light in different directions). Enantiomers generally have identical physical properties, such as melting points and boiling points, and also have identical spectroscopic properties. Enantiomers can differ from each other with respect to their interaction with plane-polarized light and with respect to biological activity. Accordingly, in some embodiments, the salts of mannuronic acid (or mannuronate) can comprise β-D-mannuronate. In some embodiments, the salts of guluronic acid (or guluronate) can comprise α-L-guluronate.

In some embodiments, alginate can be a block polymer comprising at least one or more homopolymeric regions of mannuronate (M-blocks), at least one or more homopolymeric regions of guluronate (G-blocks), at least one or more regions of alternating structure of mannuronate and guluronate (MG-blocks or GM-blocks).

The proportion, distribution and/or length of these blocks can, in part, determine the chemical and/or physical properties of an alginate gel. For example, the relative content of G and M monomers in the alginate polymers can affect, e.g., but not limited to, pore size, stability and biodegradability, gel strength and elasticity of alginate gels. Without wishing to be bound by theory, lower G content relative to M content in the alginate polymers can generally result in more biodegradable gels. Gels with higher G content alginate can generally have larger pore sizes and/or stronger gel strength relative to gels with higher M content alginate, which have smaller pore sizes and lower gel strength. In some embodiments, one or more of the alginate polymers of the alginate matrix can comprise a M-block content of at least about 10 wt %, at least about 20 wt %, at least about 30 wt %, at least about 40 wt %, at least about 50 wt %, at least about 60 wt %, at least about 70 wt %, at least about 80 wt %, at least about 90 wt % or more. In some embodiments, one or more of the alginate polymers of the alginate matrix can comprise a G-block content of at least about 10 wt %, at least about 20 wt %, at least about 30 wt %, at least about 40 wt %, at least about 50 wt %, at least about 60 wt %, at least about 70 wt %, at least about 80 wt %, at least about 90 wt % or more. In some embodiments, one or more of the alginate polymers of the alginate matrix can comprise a GM- and/or MG-block content of at least 10 wt %, at least about 20 wt %, at least about 30 wt %, at least about 40 wt %, at least about 50 wt %, at least about 60 wt %, at least about 70 wt %, at least about 80 wt %, at least about 90 wt % or more.

In some embodiments, one or more of the alginate polymers of the alginate matrix can comprise a mannuronic acid to guluronic acid (M/G) ratio of about 0.01 to about 100, or about 0.1 to about 50, or about 0.5 to about 25, or about 1 to about 20. In some embodiments, one or more of the alginate polymers of the alginate matrix can have a M/G ratio of about 1 to about 100, or about to about 50, or about 1 to about 25, or about 1 to about 20, or about 1 to about 10, or about 1 to about 5.

In some embodiments, one or more of the alginate polymers of the alginate matrix can comprise a guluronic acid to mannuronic acid (G/M) ratio of no more than 1.5 or no more than 1. For example, in some embodiments, one or more of the alginate polymers of the alginate matrix can have a G/M ratio of about 1.5. In some embodiments, one or more of the alginate polymers of the alginate matrix can have a G/M ratio of about 1. In some embodiments, one or more of the alginate polymers of the alginate matrix can have a G/M ratio of less than 1.5, including, e.g., less than 1.4, less than 1.3, less than 1.2, less than 1.1, less than 1.0, less than 0.9, less than 0.8, less than 0.7, less than 0.6, less than 0.5, less than 0.4, less than 0.3, less than 0.2, less than 0.1, less than 0.05, less than 0.01, less than 0.0075, less than 0.005, less than 0.001 or lower. In some embodiments, one or more of the alginate polymers of the alginate matrix can have a G/M ratio of less than 1, including, e.g., less than 0.9, less than 0.8, less than 0.7, less than 0.6, less than 0.5, less than 0.4, less than 0.3, less than 0.2, less than 0.1, less than 0.05, less than 0.01, less than 0.0075, less than 0.005, less than 0.001, less than 0.0001, or lower.

In some embodiments, one or more of the alginate polymers of the alginate matrix can comprise a guluronic acid to mannuronic acid (G/M) ratio of at least about 1.5 or higher. For example, in some embodiments, one or more of the alginate polymers of the alginate matrix can have a G/M ratio of about 1.5. In some embodiments, one or more of the alginate polymers of the alginate matrix can have a G/M ratio of greater than 1.5, including, e.g., greater than 2, greater than 2.5, greater than 3, greater than 3.5, greater than 4, greater than 4.5, greater than 5, greater than 6, greater than 7, greater than 8, greater than 9, greater than 10, greater than 15, greater than 20, greater than 30, greater than 40, greater than 50, greater than 60, greater than 70, greater than 80, greater than 90, greater than 100 or higher.

The average molecular weight of alginate polymers can affect, e.g., gelling time, pore size, gel strength and/or elasticity of gels. Alginate polymers can have average molecular weights ranging from about 2 kDa to 10000 kDa. Without wishing to be bound by theory, lower molecular weight of the alginate polymer can generally result in more biodegradable gels. In some embodiments, the alginate polymers of the alginate matrix can have an average molecular weight of about 5 kDa to about 10,000 kDa, or about 10 kDa to about 5000 kDa, or about 25 kDa to about 2500 kDa, or about 50 kDa to about 1000 kDa, or about 50 kDa to about 500 kDa, or about 50 kDa to about 250 kDa. In some embodiments, the alginate polymers of the alginate matrix can have an average molecule weight of about 5 kDa to about 350 kDa. In some embodiments, the alginate polymers of the alginate matrix can have an average molecule weight of about 2 kDa to about 100 kDa. In some embodiments, the alginate polymers of the alginate matrix have an average molecule weight of about 50 kDa to about 500 kDa. In some embodiments, the alginate polymers of the alginate matrix have an average molecule weight of about 50 kDa to about 300 kDa. In some embodiments, the alginate polymers of the alginate matrix have an average molecule weight of about 75 kDa to about 200 kDa. In some embodiments, the alginate polymers of the alginate matrix have an average molecule weight of about 75 kDa to about 150 kDa. In some embodiments, the alginate polymers of the alginate matrix have an average molecule weight of about 150 kDa to about 250 kDa. In some embodiments, the alginate polymers of the alginate matrix have an average molecule weight of about 100 kDa to about 1000 kDa.

In some embodiments, the alginate polymers of the alginate matrix can have an average molecular weight of less than 75 kDa or lower. In some embodiments, the alginate polymers of the alginate matrix can have an average molecular weight of at least about 75 kDa, at least about 80 kDa, at least about 90 kDa, at least about 100 kDa, at least about 110 kDa, at least about 120 kDa, at least about 130 kDa, at least about 140 kDa, at least about 150 kDa, at least about 160 kDa, at least about 170 kDa, at least about 180 kDa, at least about 190 kDa, at least about 200 kDa, at least about 250 kDa, at least about 300 kDa, or higher.

In one embodiment, the alginate polymers of the alginate matrix has an average molecular weight of about 75 kDa to about 200 kDa, with a guluronic acid to mannuronic acid (G/M) ratio of about 1. In one embodiment, the alginate polymers of the alginate matrix has an average molecular weight of about 75 kDa to about 200 kDa, with a guluronic acid to mannuronic acid (G/M) ratio of less than 1.

Without limitations, the molecular weight can be the peak average molecular weight (Mp), the number average molecular weight (Mn), or the weight average molecular weight (Mw).

The alginate can be derived from any source and/or produced by any art-recognized methods. In some embodiments, the alginate can be derived from stem and/or leaves of seaweeds or kelp. In some embodiments, the alginate can be derived from green algae (Chlorophyta), brown algae (Phaeophyta), red algae (Rhodophyta), or any combinations thereof. Examples of seaweeds or kelps include, but are not limited to, various types of Laminaria (e.g., but not limited to, Laminaria hyperborea, Laminaria digitata, and Laminaria japonica), Lessonia nigrescens, Lessonia trabeculata, Durvillaea antarctica, Ecklonia maxima, Macrocystis pyrifera, Ascophyllum nodosum, and any combinations thereof.

In some embodiments, the alginate can be a bacterial alginate, e.g., produced by a microbial fermentation using bacteria. Examples of bacteria that can be used in alginate production include, but are not limited to, Pseudomonas (e.g., Pseudomonas aeruginosa) and Azotobacter (e.g., Azotobacter vinelandii). In some embodiments, the bacteria can produce a polysaccharide polymer with a structure resembling alginate, for example, differing in that there are acetyl groups on a portion of the C2 and C3 hydroxyls.

In some embodiments, the alginate can be modified. In some embodiments, the alginate can be chemically modified. For example, a chemically modified alginate can comprise propylene glycol alginate (PGA). In some embodiments, PGA can be made by contacting a partially neutralized alginic acid with propylene oxide gas under pressure. The propylene oxide can react exothermically with the alginic acid to form a mixed primary/secondary ester.

In some embodiments, the alginate can be of clinical grade, e.g., suitable for use in vivo. In some embodiments, the alginate can be purified prior to use for cell encapsulation. See, e.g., Mallet and Korbutt, Tissue Eng Part A. 2009. 15(6):1301-1309. In some embodiments, the alginate can have low endotoxin. For example, endotoxins can be present in the alginate in an amount of no more than 150 EU/gram, no more than 100 EU/gram, no more than 75 EU/gram, no more than 50 EU/gram, no more than 25 EU/gram, no more than 20 EU/gram, no more than 10 EU/gram, no more than 5 EU/gram, no more than 1 EU/gram, no more than 0.5 EU/gram, no more than 0.1 EU/gram.

Any art-recognized alginate can be used in the methods of various aspects described herein. Examples of alginates that can be used in the compositions described herein include, without limitations, sodium alginate (sodium salt of alginic acid), potassium alginate (potassium salt of alginic acid), calcium alginate, magnesium alginate, triethanolamine alginate, PGA, and any combinations thereof. In some embodiments, soluble alginate can be in the form of mono-valent salts including, without limitation, sodium alginate, potassium alginate and ammonium alginate. In some embodiments, the alginate can be calcium alginate. In one embodiment, calcium alginate can be made from sodium alginate from which the sodium salt has been removed and replaced with calcium. Alginates described in and/or produced by the methods described in the International Patent Application Nos. WO 2007/140312; WO 2006/051421; WO2006/132661; and WO1991/007951 and U.S. Pat. No. 8,481,695 can also be used in the compositions and methods of various aspects described herein. In some embodiments, commercially-available alginates, e.g., obtained from FMC BioPolymer and Novamatrix, can also be in the compositions and methods of various aspects described herein.

Alginate generally forms a gel matrix in the presence of divalent ions and/or trivalent ions. Non-limiting examples of divalent or trivalent ions that can be used to form alginate gels include calcium ions, barium ions, strontium ions, copper ions, zinc ions, magnesium ions, manganese ions, cobalt ions, lead ions, iron ions, aluminum ions, and any combinations thereof.

In some embodiments, the alginate matrix can be covalently crosslinked. Examples of covalent crosslinking agents that can be used to covalently crosslink alginate include, but are not limited to, carbodiimides, allyl halide oxides, dialdehydes, diamines, and diisocyanates.

Eluting matrix formulations of the invention include those suitable for injection, infusion or implantation (subcutaneous, intravenous, intramuscular, intraperitoneal, intradermal, parenteral, rectal, and/or intravaginal or the like), inhalation, oral/nasal or topical administration. The formulations may conveniently be presented in unit dosage form and may be prepared by any methods well known in the art of pharmacy. The amount of CXCL12 polypeptide which can be combined in a dosage form will vary depending upon the host being treated, the particular mode of administration, e.g., injection or implantation. Formulations of this invention can be prepared according to any method known to the art for the manufacture of pharmaceuticals and can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, emulsions, creams, lotions, gels, on patches and in implants.

CXCL12 polypeptide eluting matrices of the invention encapsulate at least one cell. Encapsulated cells can include, but are not limited to, stem cells, neuronal cells, smooth or skeletal muscle cells, myocytes, fibroblasts, chondrocytes, adipocytes, fibromyoblasts, ectodermal cells, including ductile and skin cells, hepatocytes, kidney cells, liver cells, cardiac cells, pancreatic cells, islet cells, cells present in the intestine, osteoblasts and other cells forming bone or cartilage, and hematopoietic cells. In specific embodiments, the cell is an insulin producing cell, such as an islet cell (e.g., a porcine islet cell, a human islet cell or an islet cell derived in vitro, e.g., from a stem or iPS cell (e.g., SC-β cells such as human SC-β cells).

Eluting matrices of the invention are refillable CXCL12 polypeptide delivery devices implanted or otherwise inserted within a patient. For example, the matrix may comprise a needle or catheter entry port so that cells can be infused or removed without removing the matrix from the patient. Alternatively, the eluting matrices can be repeatedly administered to a subject (e.g., a “sensitized subject”), without associated immune rejection.

CXCL12 polypeptide eluting matrices of the invention are useful in the treatment of autoimmune diseases including, but not limited to, rheumatoid arthritis, uveitis, insulin-dependent diabetes mellitus, hemolytic anemias, rheumatic fever, Crohn's disease, Guillain-Barre syndrome, psoriasis, thyroiditis, Graves' disease, myasthenia gravis, glomerulonephritis, autoimmune hepatitis, and systemic lupus erythematosus.

In one embodiment, CXCL12 polypeptide eluting matrices of the invention can be formulated with islet cells or SC-β cells for use in the treatment of diabetes. Diabetes is a condition in which a person has a high blood sugar (glucose) level as a result of the body either not producing enough insulin, or because body cells do not properly respond to the insulin that is produced. In healthy persons, blood glucose levels are maintained within a narrow range, primarily by the actions of the hormone insulin. Insulin is released by pancreatic beta-cells at an appropriate rate in response to circulating glucose concentrations, the response being modulated by other factors including other circulating nutrients, islet innervation and incretin hormones. Insulin maintains glucose concentrations by constraining the rate of hepatic glucose release to match the rate of glucose clearance.

Insulin thus enables body cells to absorb glucose, to turn into energy. If the body cells do not absorb the glucose, the glucose accumulates in the blood (hyperglycemia), leading to various potential medical complications. Accordingly, diabetes is characterized by increased blood glucose resulting in secondary complications such as cardiovascular diseases, kidney failure, retinopathy and neuropathy if not properly controlled.

Two major pathophysiologies are related to increase glycemia. The first is an autoimmune attack against the pancreatic insulin-producing beta-cells (Type 1 diabetes) whilst the second is associated to poor beta-cell function and increased peripheral insulin resistance (Type 2 diabetes). Similar to Type 1, beta-cell death is also observed in Type 2 diabetes. Type 1 and often Type 2 diabetes requires the person to inject insulin.

Type 1 DM is typically characterized by loss of the insulin-producing beta-cells of the islets of Langerhans in the pancreas leading to insulin deficiency. This type of diabetes can be further classified as immune-mediated or idiopathic. The majority of Type 1 diabetes is of the immune-mediated nature, where beta-cell loss is a T-cell mediated autoimmune attack. Type 2 DM is characterized by beta-cell dysfunction in combination with insulin resistance. The defective responsiveness of body tissues to insulin is believed to involve the insulin receptor. Similar to Type 1 diabetes an insufficient beta cell mass is also a pathogenic factor in many Type 2 diabetic patients. In the early stage of Type 2 diabetes, hyperglycemia can be reversed by a variety of measures and medications that improve insulin secretion and reduce glucose production by the liver. As the disease progresses, the impairment of insulin secretion occurs, and therapeutic replacement of insulin may sometimes become necessary in certain patients.

Regulatory T-cells are a subset of CD4+ T cells originated from the thymus, which are generally known to play a significant role in maintenance of tolerance. Regulatory T-cells actively play a role in immune modulation, and suppress alloimmune responses of transplant rejection (C. A. Piccirillo. Cytokine 43, 395 (September, 2008); G. Xia et al. Translational research: the journal of laboratory and clinical medicine 153, 60 (February, 2009); K. J. Wood. Transplantation proceedings 43, 2135 (July-August, 2011); and G. Feng et al. Transplantation 86, 578 (Aug. 27, 2008)). Regulatory T-cells prevent murine autoimmune diabetes and control autoreactive destruction of transplanted islets (M. J. Richer et al. PloS one 7, e31153 (2012) and D. R. Tonkin et al. Immunol 181, 4516 (Oct. 1, 2008)). Islet transplantation represents a potentially curative approach to diabetes, however, in previous studies of islet transplantation, systemic immune suppression could not achieve long-term control of blood glucose levels due to immune-mediated rejection of transplanted islets. Incorporation of CXCL12 into a matrix encapsulating transplanted islets provides both a physical and a biological barrier to cell-mediated and humoral anti-islet immunity. The CXCL12 polypeptide repels effector T-cells and recruits immune-suppressive regulatory T-cells, while reducing or eliminating the need for systemic immunosuppression. Accordingly, in one embodiment, CXCL12 polypeptide eluting matrices of the invention are useful for the regeneration, replacement or substitution (partial or wholly) of at least part of the pancreas of a patient deficient in pancreatic cells, particularly beta-cells without concomitant immunosuppression. Any patient whose pancreas does not produce sufficient insulin, or indeed any insulin, may benefit from such therapy. Insufficient insulin production includes the production of lower levels of insulin compared to a normal (healthy) subject; but it also includes subjects who produce insulin levels that are comparable to normal (healthy) subjects but who require higher insulin levels, for example due to insulin resistance, excessive food consumption, morbid obesity and the like.

CXCL12 polypeptide eluting matrices of the invention selectively recruit regulatory T-cells, thereby prolonging survival of the implanted matrix and providing protection from immune destruction in even a sensitized host. Accordingly, CXCL12 polypeptide eluting matrices of the invention are retrievable, and can be repeatedly administered, if desired, without immune system rejection. Furthermore, CXCL12 polypeptide eluting matrices of the invention provide sustained islet survival and continuous production of CXCL12 polypeptides from the encapsulated islets, for at least about 1 month to about 2 years. During this time, the fasting serum concentration of glucose in the subject is maintained at a blood level of between about 80 mg/dl and about 120 mg/dl. According, CXCL12 polypeptide eluting matrices of the invention are particularly useful in the treatment of diabetes.

With respect to β-cells, in particular SC-β cells, the presence of CXCL12 is demonstrated to provide multiple beneficial effects, both before and after implantation in a subject. These include, without limitation, increasing survival, increasing functional maturation, improving glucose-stimulated insulin secretion, improving glucose responsiveness, decreasing apoptosis, decreasing the item to achieve normoglycemia, and increasing the length time for which normoglycemia is maintained. The presence of CXCL12 may increase survival of implanted SC-β cells such that the cells survive in a subject for at least 30 days, e.g., at least 45, 60, 100, 125, or 150 days or more. The presence of CXCL12 in a capsule containing SC-β cells also increases the immunoisolation of implanted cells, e.g., SC-β cells, and capsules containing the cells. This not only increases the survival of the cells but inhibits the immune response to the capsules, e.g., fibrotic pericapsular overgrowth of the capsules.

In certain embodiments, the beneficial effects of CXCL12 may be dose dependent or inverse dose dependent. In some embodiments, lower doses of CXCL12 (e.g., about 0.2 μg/ml) may be more effective than higher doses (e.g., about 2 μg/ml), e.g., with respect to maturation and survival of SC-β cells. In other embodiments, higher doses of CXCL12 (e.g., about 2 μg/ml) may be more effective than lower doses (e.g., about 0.2 μg/ml), e.g., with respect to immunoisolation and prevention of fibrosis.

Thus, one aspect of the invention relates to a method of treating diabetes in a subject in need thereof, comprising delivering to the subject an effective amount of the composition of the invention, thereby treating diabetes.

Another aspect of the invention relates to a method of accelerating the normalization of hyperglycemia in a subject in need thereof, comprising delivering to the subject an effective amount of the composition of the invention, thereby accelerating the normalization of hyperglycemia.

A further aspect of the invention relates to a method of preventing fibrotic pericapsular overgrowth of microcapsules in a subject, comprising delivering to the subject an effective amount of the composition of the invention, thereby preventing fibrotic pericapsular overgrowth of the microcapsules.

An additional aspect of the invention relates to a method for enhancing a response against diabetes in a subject in need thereof, comprising delivering to the subject an effective amount of the composition of the invention, thereby enhancing the response against diabetes.

The present invention is additionally described by way of the following illustrative, non-limiting Examples that provide a better understanding of the present invention and of its many advantages.

### Examples

The recent breakthroughs in generating functional 13 cells from human inducible pluripotent stem cells (iPSCs) in vitro, so-called SC-β cells, potentially make β-cell replacement therapy to cure diabetes a practicality. However, potential immune rejection and limited duration of survival and function post-transplantation are major hurdles to their clinical application.

The chemokine stromal cell-derived factor 1 (SDF-1), also known as CXCL12, exerts local anti-inflammatory and immunosuppressive effects via multiple mechanisms and has pro-survival and insulinotropic effects on β-cells. Signaling by CXCL12 is also an essential component of pancreatic β-cell development, maturation, survival and function. Within the native pancreatic islet, CXCL12 and its cognate receptors (CXCR4 and CXCR7) are expressed, with the chemokine providing pro-survival, regenerative and immunoregulatory signals to the β-cells. Accordingly, mice transgenically expressing CXCL12 in their β-cells are resistant to streptozotocin (STZ) induction of hyperglycemia, while the CXCL12-CXCR4 signaling axis within the islet microenvironment prevents autoimmune diabetes. It is worth noting that tumor cells commonly exploit overexpression of this chemokine to provide them with an immune privilege and survival advantage.

Like most chemokines, CXCL12 has a net positive charge, which enables it to bind with high affinity to the negatively charged polysaccharide anions of alginate thereby generating a chemokine gradient to modulate local immune responses. We previously demonstrated long-term survival, function and glycemic control by transplanted murine alloislets engineered to express CXCL12 and porcine xenoislets in alginate microcapsules that elute the chemokine without systemic immunosuppression.

Here, we harness the immunoregulatory and pro-survival effects of CXCL12 to immunoisolate and support long-term survival and function of type 1 diabetes patient derived SC-β cells in alginate microcapsules to achieve restoration of long-term normoglycemia in sensitized immune competent C57BL/6 diabetic mice without systemic immunosuppression. We show that CXCL12 enhances glucose-stimulated insulin secretion, induces expression of key β-cell function genes in SC-β cell clusters and promotes their survival. Finally, we demonstrate that co-encapsulation of recombinant human CXCL12 with SC-β cell clusters into alginate microcapsules accelerates the normalization of hyperglycemia and prevents fibrotic pericapsular cellular overgrowth to prolong SC-β cell survival and long-term normoglycemia in sensitized diabetic mice. These preliminary findings rationalize studies in nonhuman primates with this SC-β cell encapsulation technology and potentially a human clinical trial.

### Materials and Methods

Derivation and Culture of SC-β Cell Clusters.

SC-β clusters were differentiated from human inducible pluripotent stem cells as previously described. The cell clusters were cultured in modified CMRL medium containing 10% FBS and 1% penicillin/streptomycin in spinner flasks on a stir platform rotating at 100 rpm in a humidified, 37° C., 5% CO₂ cell culture incubator. Three-quarters of the culture medium was replaced every 48 h.

Production of Alginate Microcapsules Containing SC-11 Cell Clusters with and without CXCL12.

The production of alginate microcapsules was carried out using a BUCHI Encapsulator B-395 Pro Sterile Microcapsule Production System that was set up in a type II class A2 biological safety cabinet. A sterile 1.6% w/v sodium alginate solution was first prepared by dissolving ultra-pure low viscosity mannuronate sodium alginate (PRONOVA™ UP LVM) in 150 mM NaCl solution, stirred overnight at 4° C. and filtered through a 0.8/0.2-μm sterile syringe filter (PALL Life Sciences). Pelleted stage 6 differentiated SC-β clusters that are 6-7 days old (100-250 μm in diameter) together with recombinant murine CXCL12-α (PeproTech) were homogeneously suspended in the 1.6% w/v sodium alginate solution to yield a density of 2000 SC-β clusters per 1.0 ml and either 0.0, 0.2 or 2.0 μg/ml of CXCL12. The homogeneous mixture was loaded into a 60-ml Luer-lock syringe (Thermo Fisher Scientific) and attached to an air-dripping nozzle system of the BUCHI Encapsulator B-395 Pro apparatus. The mixture was pumped through a 400-μm nozzle into a reaction chamber containing 100 mM CaCl₂ cross-linking solution that is being stirred at 100 mBar using the following encapsulation settings: 1013 Hz internal vibration frequency of the encapsulator, 2.05 mBar air pressure and 1.8 ml/min syringe pump rate. The alginate microcapsule droplets from the nozzle were allowed to gel in the CaCl₂ solution for 5 min, washed with CMRL culture medium, and subsequently cultured in the same medium until use. The size of microcapsules immediately after CaCl₂ cross-linking ranged from 600-700 μm in diameter and increased by 18% in diameter after overnight culture in CMRL medium.

Glucose-Stimulated Insulin Secretion (GSIS) Assays.

To determine the effect of CXCL12 on glucose-stimulated insulin secretion (GSIS), 10-20 SC-β clusters or their equivalent in alginate microcapsules (with or without CXCL12) were cultured for 24 h overnight in culture medium containing 2 mM glucose and indicated concentrations of CXCL12. The clusters or their microcapsules were then washed with Krebs buffer, and subjected to alternating 30-min incubations in 2 mM and 20 mM glucose in Krebs buffer with the supernatants collected following each incubation. After glucose stimulations, the cells were pelleted and those in alginate microcapsules retrieved by dissolving alginate in sodium citrate containing EDTA. Total protein was extracted from the cells with total protein extraction buffer containing protease and phosphatase inhibitors (RIPA Lysis buffer, Thermo Fisher Scientific). The concentration of human C-peptide in the supernatants and the total protein content of the cell extracts were determined using human insulin C-peptide ELISA (R&D Systems) BCA test (Thermo Fisher Scientific), respectively. The insulin C-peptide secreted was normalized to the total protein content of the cell extract. GSIS indices were determined by dividing the amount of C-peptide secreted following incubation in high glucose (20 mM) by that following incubation with low glucose (2 mM).

Animals and Animal Studies.

The animal studies protocol was approved by the IACUC of the Massachusetts General Hospital (MGH). Female C57BL/6 mice (6 weeks old) were purchased from the Jackson Laboratories (Bar Harbor, Me., USA) and housed and fed according to standard protocol.

Induction of Diabetes.

To induce insulin-dependent diabetes, mice were injected intraperitoneally with 250 mg/Kg-body weight of streptozotocin (STZ) dissolved in 114 mM sodium citrate (pH 4.5). For inclusion in our studies, animals were considered diabetic enough when they had at least two consecutive blood plasma glucose readings of ≥400 mg/dl. To sensitize mice, SC-β clusters we disrupted by 5 cycles of freeze-thaw in liquid nitrogen and 37° C. water bath, respectively. Approximately 1×10⁶ cells were then injected intraperitoneally into diabetic mice at least 5 days before transplanting with microcapsules containing SC-β clusters.

Transplantation Procedure.

Anesthesia was induced in the animal by a subcutaneous injection of a cocktail comprising ketamine and xylazine (80 mg/kg and 10 mg/kg body, respectively). The abdomen was shaved and sterilized with Betadine (Povidone Iodine solution USP 10%), a 0.5-mm incision along the midline abdominal skin made, and the peritoneal lining exposed by blunt dissection. While grasping the peritoneal wall with forceps, a 0.5-1.0 mm incision was made along the linea alba. Through the incision, the microcapsules (300 or 400) were implanted into the peritoneal cavity using a sterile spatula. The incisions were sutured with Ethilon 5-0 nylon suture and wipe with Betadine.

Intraperitoneal Glucose Tolerance Test (IPGTT), Glucose, and C-Peptide Monitoring.

IPGTT test was preceded by a 6 h fast (from 8:00 AM to 2:00 PM). Mice were then injected intraperitoneally with 2 g/Kg-body weight of glucose in PBS. The levels of blood plasma glucose were determined before glucose administration (0 min) and at 15, 30, 60, 90 and 120 min after glucose administration using ˜0.5-μl blood from a tail vein prick with a glucometer kit (Accu-Chek Performa Glucometer). The non-random blood plasma glucose levels of mice were monitored on regular bases (8:00-10:00 AM every 2-3 or weekly basis) following transplantation via the same method. To determine serum C-peptide levels, blood (100-200 μl) was drawn from mice under anesthesia via retro-orbital bleed at designated time points and serum isolated by centrifugation at 2000 rpm for 15 min after clotting for 30 min Human insulin C-peptide and murine insulin C-peptide concentrations were then determined with their respective ELISA kits per the manufacturers' protocols (R&D Systems).

Flow cytometry. To determine the proportion of cell types within SC-β clusters, 100 clusters were dispersed into single cell suspensions using TrypLE Express without phenol and washed once with 2% FBS in PBS. Cells were fixed in 4% PFA on ice for 30 min and washed 2× with a blocking buffer (BB) comprising 5% FBS and 0.1% Triton X-100 in PBS. The cells were incubated with primary antibodies against C-peptide (mouse anti-C-peptide) and NKX6.1 (rabbit anti-NKX6.1) overnight at 4° C. Cells were washed 2× with BB and incubated with corresponding fluorophore-conjugated secondary antibodies (donkey anti-mouse IgG Alexa Fluor 488, donkey anti-rabbit IgG Alexa Fluor 594 for anti-C-peptide and anti-NKX6.1 and anti-glucagon antibodies, respectively) in BB for 1 h at room temperature (RT). Cells were washed 3× with PBS and re-suspended in 500 μl of 2% FBS in PBS for FACS analysis.

Apoptotic Caspase-3 Activity Assay.

About 80-100 SC-β clusters were seeded into 12-well plates and treated with 0.0, 10.0, 100.0 and 1000.0 ng/ml recombinant human CXCL12-α for 4 h. A cytokine cocktail comprising IL-1β (0.05 μg/ml), TNF-α (0.25 μgimp and IFN-γ (1.8 μg/ml) was then added and incubated for a further 48 h. A biotinylated caspase-3 inhibitor was added to cells for 1 h to specifically bind active caspase-3. The clusters were harvested and cell extracts subjected to human active caspase-3 ELISA using Human Active Caspase-3 Immunoassay kit (R&D Systems) per the manufacturer's instructions. The total protein concentration in each treatment cell extract was determined with a BCA test kit (Thermo Fisher Scientific) and caspase-3 activity normalized to the total protein concentration for each treatment.

RT-qPCR.

After appropriate treatments, total RNA was extracted from samples using the Qiagen RNeasy Mini Kit. Complementary DNA (cDNA) was reversed transcribed from 1.0-2.0 μg of total RNA using SuperScript III First-Strand Synthesis SuperMix for RT-qPCR (Thermo Fisher Scientific). The cDNA was amplified by RT-qPCR using The Applied Biosystems® StepOne™ Real-Time PCR Systems (Thermo Fisher Scientific). The PCR products of the housekeeping gene Gapdh served as internal controls and threshold cycle numbers (Ct) for each sample normalized to the Ct values for GAPDH. The mRNA levels of test samples were expressed relative to control samples using the 2^{-ΔΔct} method. The forward and reverse primers used for the RT-qPCR are listed in Table 2.

TABLE 2

| Primer | Sequence (5′ to 3′) |
|---|---|
| hGAPDH_F | TGCACCACCAACTGCTTAGC |
| | SEQ ID NO: 7 |
| hGAPDH_R | GGCATGGACTGTGGTCATGAG |
| | SEQ ID NO: 8 |
| hPCSK1_F | AAGCAAACCCAAATCTCACCTGGC |
| | SEQ ID NO: 9 |
| hPCSK1_R | TCACCATCAAGCCTGCTCCATTCT |
| | SEQ ID NO: 10 |
| hTCF7L2_F | TCGCCTGGCACCGTAGGACA |
| | SEQ ID NO: 11 |
| hTCF7L2_R | GGATGCGGAATGCCCGTCGT |
| | SEQ ID NO: 12 |
| hWnt5a_F | CAACTGGCAGGACTTTCTCA |
| | SEQ ID NO: 13 |
| hWnt5a_R | TTCTTTGATGCCTGTCTTCG |
| | SEQ ID NO: 14 |
| hFlattop_F | TTGCCAACGATCGTGGTCAT |
| | SEQ ID NO: 15 |
| hFlattop_R | GGATCATGGGGCTTGCCTAA |
| | SEQ ID NO: 16 |
| hPDX1_F | ACCAAAGCTCACGCGTGGAAA |
| | SEQ ID NO: 17 |
| hPDX1_R | TGATGTGTCTCTCGGTCAAGTT |
| | SEQ ID NO: 18 |
| hGCK_F | TGGACCAAGGGCTTCAAGGCC |
| | SEQ ID NO: 19 |
| hGCK_R | CATGTAGCAGGCATTGCAGCC |
| | SEQ ID NO: 20 |

Hematoxylin and Eosin and Immunofluorescence Staining.

The SC-β clusters and/or their microcapsules (retrieved and fresh microcapsules pre-implantation) samples were fixed in 4% PFA or 10% zinc formalin overnight at 4° C., washed with deionized water and transferred to 70% ethanol for further processing. The samples were embedded in paraffin blocks (LEICA EG1160) and cut into 0.5-μm sections with an automated rotary microtome (LEICA CM3050 S Cryostat). The paraffin-embedded sections were deparaffinized in xylene for 10 min and rehydrated by incubation for 3 minutes each in serial concentrations of 100%, 95%, 70% and 50% ethanol and washed in deionized water. To retrieve antigens after deparaffinization, the sections slides were immersed in a 10 mM sodium citrate (pH 6.0) solution that was brought to boiling point in a microwave (full power for 2 minutes), incubated at sub-boiling point for 10 minutes and allowed to cool for 30 minutes at room temperature. The sections were permeabilized by 2 incubations in 1% FBS+0.4% Triton X-100 in PBST for 10 min and blocked with 5% FBS in PBST for 30 min at RT. Samples were stained with H&E. For immunofluorescence staining, sections were incubated with primary antibodies as described for flow cytometry overnight at 4° C. The sections were washed twice with PBS and incubated with secondary antibodies diluted in 1% FBS in PBST for 1 h at RT. Sections were then washed twice with 1% FBS in PBST, 10 min per wash. The sections were then counterstained with DAPI and covered with coverslips for microscopy. Both H&E and immunofluorescence sections were imaged at 10× and/or 20× using a Spinning Disk confocal microscope (Yokogawa Spinning Disk Confocal/TIRF system).

Statistical Analysis.

Data are presented as the mean±SEM from at least three biological replicates. Statistical analysis was done using GraphPad Prism (GraphPad Software 7.02, Inc., La Jolla, Calif., USA). Differences among groups were assessed by one-way ANOVA with post hoc Bonferroni test to identify the significance of differences among means, defined as p<0.05. Sample size was predetermined based on the variability observed in preliminary data.

### Results

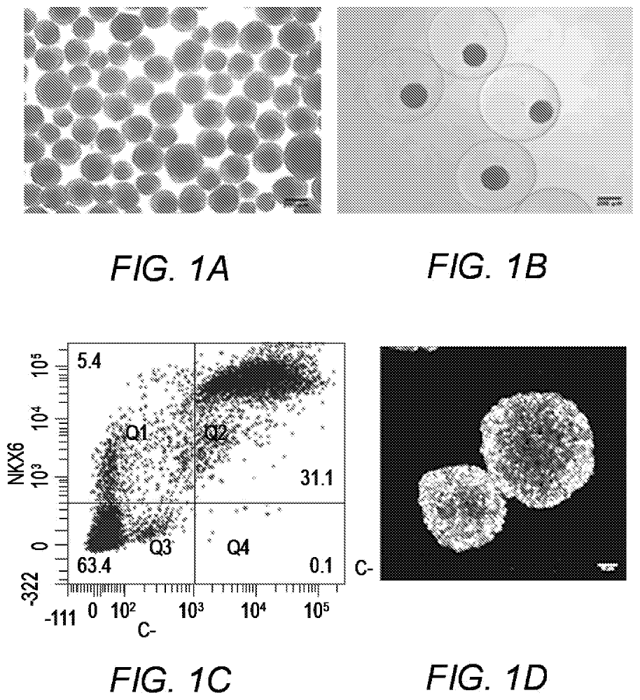

Characteristics of SC-β cell clusters and alginate microcapsules. The SC-13 cell clusters used in this study were derived from T1D patient iPSCs as previously described. The diameter of alginate microcapsules containing SC-β cell clusters (150-250 μm diameter; FIG. 1A) ranged from 650-700 μm (FIG. 1B). Most of the microcapsules contained a single SC-β cell cluster, with a few (˜25%) containing 2-3 clusters or were blank. On average, the fraction of human insulin C-peptide-positive cells in all batches of SC-β cell clusters used in this study, as determined by intracellular immunostaining flow cytometry and confocal microscopy, varied from 30-60% (FIGS. 1C-1D).

CXCL12 Enhances Glucose-Stimulated Insulin Secretion (GSIS) by SC-β Cell Clusters.

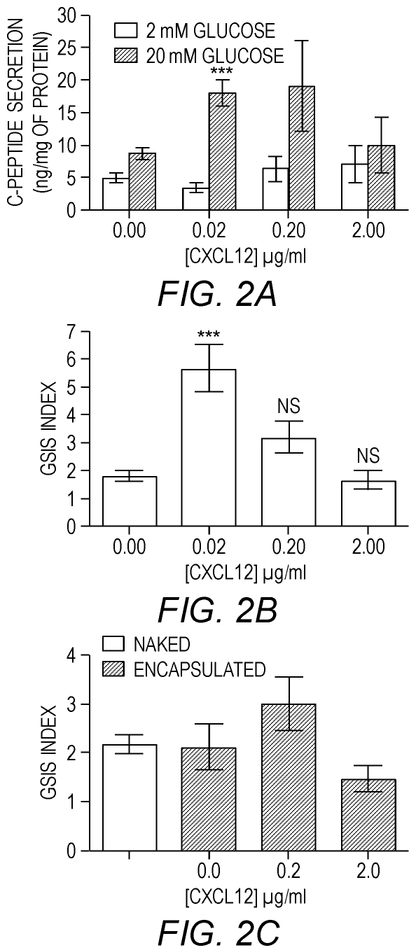

The key function of β cells is to maintain glucose homeostasis by secreting insulin in response to glucose stimulation. We first determined the impact of CXCL12 (with and without alginate microencapsulation) on insulin C-peptide secretion by SC-β cell clusters. The SC-β cell clusters were encapsulated with 0.0, 0.2 and 2.0 μg/ml CXCL12 while the naked clusters cultured with 0, 0.02, 0.2 and 2.0 μg/ml CXCL12. The microcapsules or naked clusters were then subjected to glucose stimulations after 24 h overnight culture. CXCL12 exerted an inverse dose-dependent enhancement of insulin C-peptide secretion in both naked and encapsulated clusters (FIGS. 2A-2C). Treatment with 0.02 μg/ml CXCL12 on naked clusters resulted in significant increase in insulin C-peptide secretion compared with control treatment, causing a 5.6-fold increase in C-peptide secretion following stimulation with high glucose (FIG. 2B; p<0.01) compared with ˜2-, ˜3.2- and ˜1.7-fold increase in C-peptide secretion for non-treatment, 0.2 and 2.0 μg/ml treatments, respectively. Overall, alginate-encapsulation did not affect the GSIS of SC-β cells, and CXCL12 exerted a similar effect on GSIS when co-encapsulated with SC-β cell clusters in alginate microcapsules as found with the naked clusters (FIG. 2C). Thus, incorporation of 0.2 and 2.0 μg/ml CXCL12 with SC-β cell clusters in alginate microcapsules resulted in ˜3.1- and ˜1.5-fold increase in insulin C-peptide secretion following glucose stimulation compared with ˜2.1 for 0.0 μg/ml CXCL12 treated microcapsules or naked clusters (FIG. 2C).

CXCL12 Induces Expression of β-Cell Function Genes and Promotes SC-β Cell Cluster Survival.

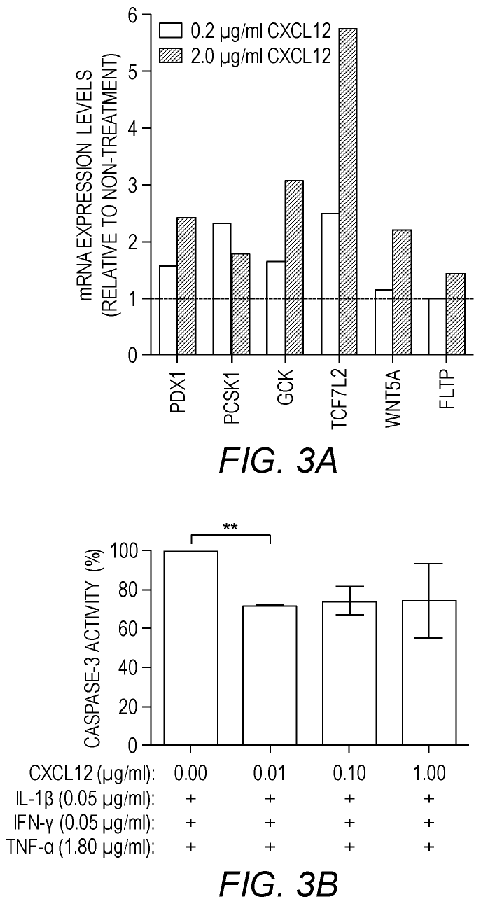

Enhanced β-cell function often promotes their survival. Having observed that CXCL12 enhances the insulin secretion function of SC-β cells, we determined the effect of CXCL12 on the expression levels of key genes associated with β-cell function by RT-qPCR and caspase-3 activity to assess their apoptosis in response to treatment with key cytokines that induce β-cell death during T1D (IL-1β, TNF-α and IFN-γ). A 24-h exposure of SC-β cell clusters to CXCL12 increased the mRNA transcripts levels of Gck (encodes glucokinase, the enzyme that controls glucose uptake and glycolysis rate), Pdx1, Pcsk1 (pro-insulin processing enzyme), Tcf712, Wnt5a, and Fltp (FIG. 3A). These are genes that are known to promote islet β-cell function and survival. For instance, several studies indicate that the WNT pathway of which Tcf712 is a component, promotes 13 cell survival and function. Also, activation of Wnt/PCP pathway, of which Fltp is a downstream effector, enhances human β-cell maturation in vitro. In summary, we have demonstrated that exogenous administration of CXCL12 could improve functional maturation of SC-β cells and their glucose responsiveness.

To confirm the pro-survival effect of CXCL12 on SC-β cell clusters, sc-β cell clusters were pretreated with varying concentrations of recombinant CXCL12 and then incubated with a cocktail of the key cytokines that induce β-cell apoptosis during the pathogenesis of T1D, including IL-1β, IFN-γ and TNF-α. As shown in FIG. 3B, pretreatment with CXCL12 prevented apoptosis of SC-β cell clusters relative to control treatment as marked by reduced caspase 3 activity. In line with decreased function associated with the high doses of CXCL12, we observed that lower doses of CXCL12 elicited more pro-survival effects compared with higher doses. Thus 0.01 μg/ml CXCL12 produced more anti-apoptotic effect compared with 1.0 μg/ml CXCL12 (FIG. 3B).

Co-Encapsulation of SC-β Cell Clusters with CXCL12 Provides Enhanced Immunoisolation In Vivo.

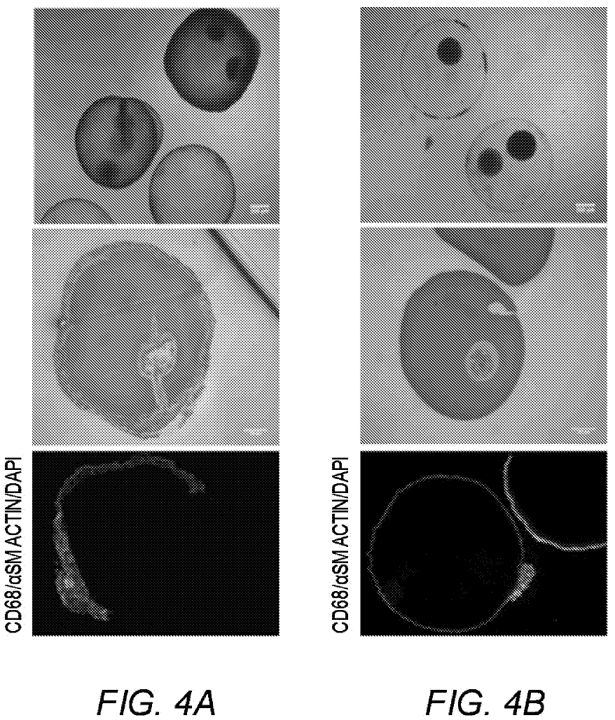

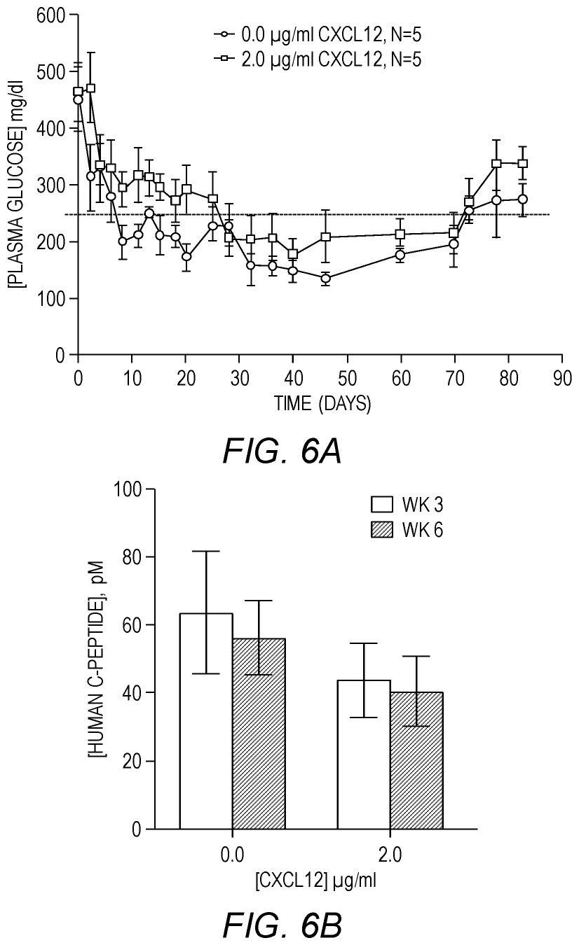

Having demonstrated that CXCL2 enhances the function and survival of SC-β cells in vitro, we next explored the ability of CXCL12 to provide immunoisolation for alginate-encapsulated SC-β cell clusters in immune-competent diabetic C57BL/6 mice. The C57BL/6 mouse is known to elicit a robust foreign body reaction to biomaterial implants that mimics the foreign body response in humans. For this study, mice were considered diabetic and treated with the encapsulated SC-β cell clusters if they showed two consecutive plasma glucose readings ≥400 mg/dl whereas the animals were considered to have returned to hyperglycemia post-transplant if they showed two consecutive blood plasma glucose readings ≥250 mg/dl. We have previously demonstrated that CXCL12 exerts chemorepellent and immunosuppressive effects at higher doses. We therefore, chose to compare the capacity of alginate microcapsules incorporating a higher concentration of CXCL12 (2.0 μg/ml) with microcapsules without the chemokine to provide immunoisolation for the encapsulated SC-β cell clusters. Immune-competent STZ-induced diabetic C57BL/6 mice were implanted with microcapsules±2.0 μg/ml CXCL12 containing the equivalent of 300 SC-β cell clusters. Implantation of the equivalent of 300 SC-β cell clusters in alginate microcapsules with and without CXCL12 both restored normoglycemia up to 70 days without overt rejection (plasma glucose concentrations <250 mg/dl) in 100% of mice in each treatment group (FIG. 6A). Also, human C-peptide in the sera of all mice at weeks 3 and 6 post-transplantation were detected, and the levels were not significantly different between treatment groups (FIG. 6B). We retrieved microcapsules at week 12 post-transplantation, when all the mice had returned to a hyperglycemic state (plasma glucose concentrations >250 mg/dl), and analyzed the immune responses to the implanted microcapsules. The foreign body response to biomaterial implants such as alginate microspheres is often characterized by fibrotic pericapsular cellular overgrowth, containing macrophages and αSMactin. Both phase contrast microscopy and hematoxylin and eosin (H&E) staining on the retrieved showed that microcapsules without CXCL12 were characterized by extensive pericapsular overgrowth and necrosis of the encapsulated SC-β cell clusters whereas those that incorporated 2.0 μg/ml CXCL12 had little pericapsular overgrowth (FIGS. 4A-4B). Intriguingly, retrieved microcapsules that did not contain SC-β cell clusters (blank microcapsules) showed little pericapsular cellular overgrowth, irrespective of whether CXC12 was incorporated or not. As mentioned, the foreign body response to biomaterial implants is often characterized by a fibrotic pericapsular cellular overgrowth, with macrophages playing a key role and αSMactin being a characteristic feature of fibrosis. As shown in the bottom panels of FIGS. 4A-4B, the pericapsular overgrowth on microcapsules without CXCL12 intensively stained positive for CD68 and αSMactin, markers for macrophages and fibrosis, respectively, compared with microcapsules containing 2.0 μg/ml CXCL12. These findings suggest that the pericapsular cellular overgrowth was triggered by the encapsulated SC-β cell clusters, likely because of the production and secretion of inflammatory cytokines and/or damage-associated molecular patterns (DAMPs) and not due to the alginate biomaterial.

Co-Encapsulation of SC-β Cell Clusters with CXCL12 Prolongs their Survival and Function to Restore Long-Term Normoglycemia in Sensitized Immune-Competent Diabetic Mice.

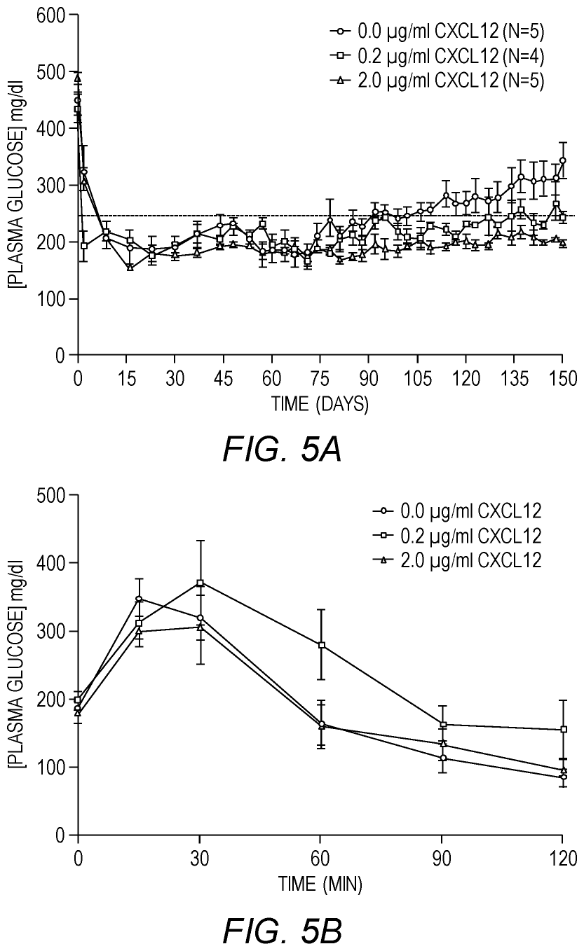

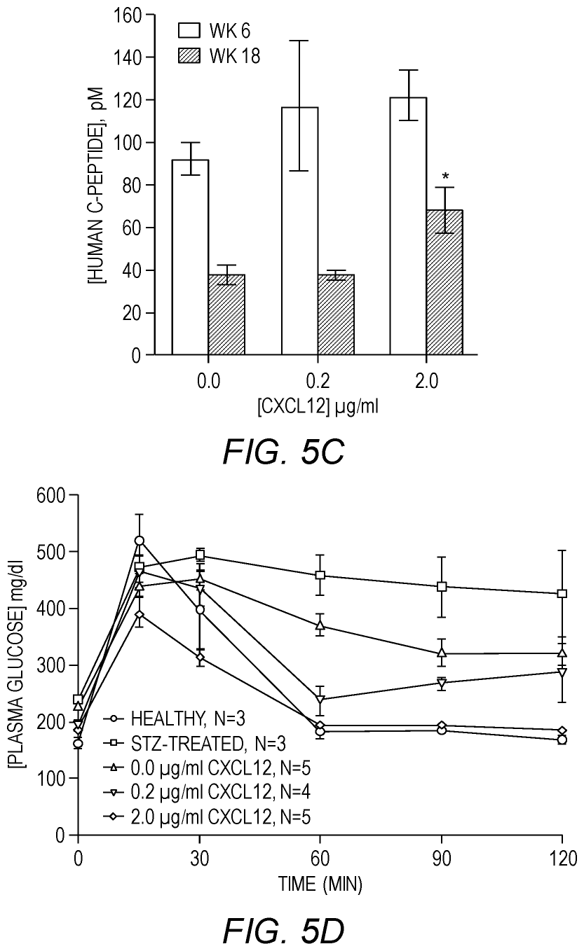

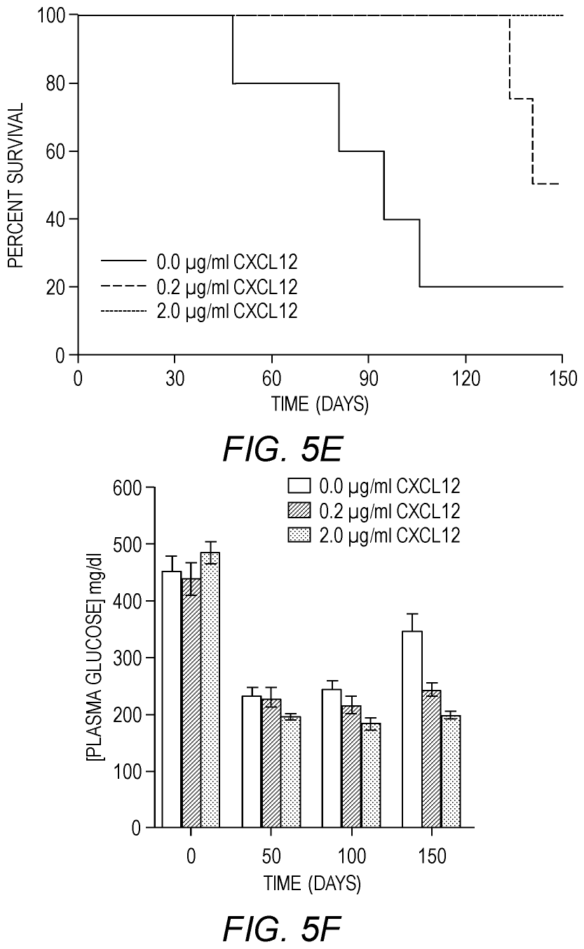

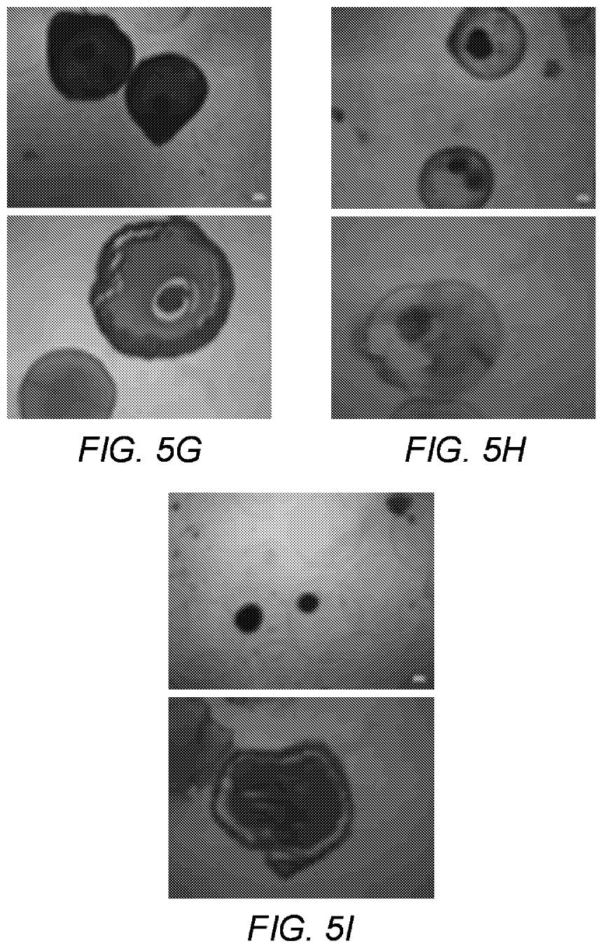

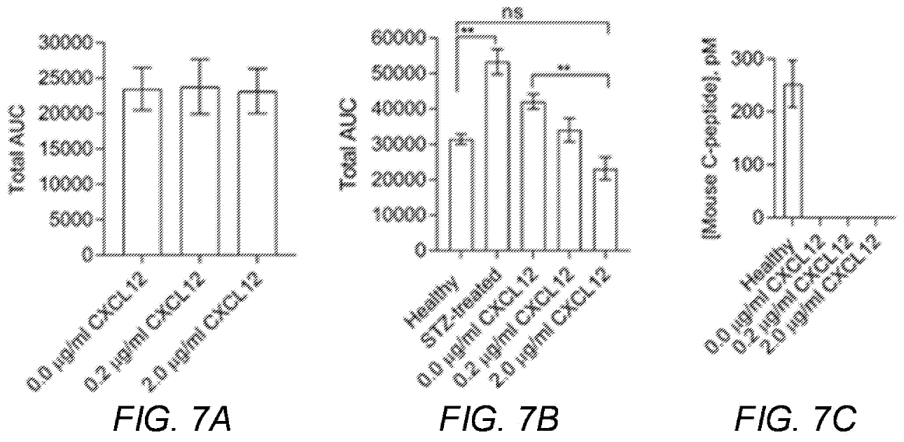

The size of alginate microcapsules influences the foreign body response, with large microcapsules (≥1.5 mm) reducing the foreign body response. Vegas et al., previously reported that SC-β cell clusters in unmodified alginate microcapsules, irrespective of the capsule size, were rejected within 30 days post-transplantation. In our preliminary exploration, we observed significant differences in the immune responses to the implanted microcapsules. We postulated that the inability of the SC-β cell clusters to restore robust and prolonged normoglycemia was due to insufficient SC-β cells to start with, and that significant differences would be observed in a more robust immune system. To test this hypothesis, we immunized the immune-competent C57BL/6 mice against the SC-β cells by injecting the mice with disrupted SC-β cells intraperitoneally a week prior to implantation with the microcapsules. The number of implanted SC-β cell clusters in the microcapsules was also increased to 400 per mouse. Also, because we observed increased function and insulin secretion by the SC-β cell clusters following treatment with 0.2 μg/ml CXCL12 in vitro, we included SC-β cell clusters in microcapsules containing 0.2 μg/ml CXCL12. Mice were implanted with microcapsules 24 h after encapsulation of the SC-β cell clusters. In congruence with the increased insulin secretion observed in vitro, normoglycemia was restored in mice implanted with microcapsules incorporating 0.2 μg/ml CXCL12 within 2 days post-transplantation whereas those implanted with microcapsules without CXCL12 or with 2.0 μg/ml CXCL12 became normoglycemic only after one week (FIG. 5A). Three weeks post-implantation when all mice had become normoglycemic, the mice were challenged with an intraperitoneal injection with a bolus 2 g/kg body weight of glucose. At this stage, all mice responded to the intraperitoneal blood glucose tolerance tests (IPGTT), with blood glucose levels normalizing within 60-90 minutes (FIG. 5B). There were not significance differences for the area under the curve (AUC) for glucose levels following IPGTT among treatment groups (FIG. 7A). We also detected significant fasting blood serum human C-peptide levels in all treated mice 6 weeks post-implantation without significant differences among groups (FIG. 5C). However, by 150 days post-implantation, only mice transplanted with 2.0 μg/ml CXCL12-containing microcapsules showed a stronger capacity to maintain glucose homeostasis in response to the intraperitoneal bolus glucose challenge similar to healthy control mice (FIG. 5D). The glucose AUC for this group of mice was significantly lower than for those carrying microcapsules with 0.2 μg/ml CXCL12 or without CXCL12 or STZ-treated diabetic mice (FIG. 7B). Accordingly, the serum human C-peptide levels in the mice carrying microcapsules with 2.0 μg/ml CXCL12 were significantly higher than those carrying microcapsules without CXCL12 or with 0.2 μg/ml CXCL12 (FIG. 5C). Based on our criteria for rejection or reversal to hyperglycemia, 80% and 50% of mice carrying SC-β cell clusters in microcapsules without CXCL12 and 0.2 μg/ml CXCL12, respectively did not survive up to 150 days whereas 100% of those carrying microcapsules with 2.0 μg/ml CXCL12 did (FIG. 5E, p<0.01 log-rank test). Upon determination of mouse C-peptide levels using a kit that detects only mouse C-peptide, we did not detect mouse C-peptide levels (levels were below the detection limits) in the treated mice carrying the SC-β cells whereas significant levels were detected in healthy control mice (FIG. 7C). These observations suggest that the mice depended on the transplanted human cells to maintain glucose homeostasis. We retrieved microcapsules from the mice after 150 days of transplantation and analyzed them for fibrotic responses. By both phase contrast microscopy and H&E staining, microcapsules containing SC-β cells without CXCL12 were typified by extensive pericapsular overgrowth and necrotic cells followed by those incorporating 0.2 μg/ml CXCL12 whereas those containing 2.0 μg/ml showed little pericapsular cellular overgrowth. Taken together, we have demonstrated that alginate co-encapsulation of SC-β cell clusters with 2.0 μg/ml CXCL12 prevents the fibrotic response and promotes survival and function of the cells to restore long-term normoglycemia.

### DISCUSSION

CXCL12, like other chemokines, is a small molecular weight positively charged protein that binds with high affinity to anionic extracellular glycosaminoglycans (GAGs) such as heparin in the native cell. The anionic polysaccharide chains within alginate microcapsules can serve as substitutes for the native mammalian negatively charged GAGs, providing multivalent binding sites to support reversible loading and slow release of CXCL12. This could result in sustained release over a period of 3 weeks, covering the period when the FBR to implants occurs. Chemokines and their receptors are key regulators of the local inflammatory response by directly modulating the cellular infiltration around implants. Moreover, CXCL12 induces M2 type macrophages, which could resolve inflammatory responses. In this study, we exploited the β-cell pro-survival and local anti-inflammatory and immunosuppressive effects of CXCL12 in conjunction with its desirable binding and release kinetics from alginate to achieve enhanced immunoisolation and survival of SC-β cells and long-term glycemic control in sensitized mice. SC-β cells have the potential to provide a cure for T1D considering that these cells can be generated in vitro in scalable quantities. We demonstrated that CXCL12 promotes the survival and function of SC-43 cells and provides potent immunoisolation when encapsulated with SC-β cells in simple alginate microcapsules. This has prompted us to start a similar study in NHPs.

A previous study employed modifications of the composition and size of alginate microcapsules to reduce the foreign body immune response to encapsulated SC-β cells to achieve long-term glycemic control. However, this requires relatively large microcapsule sizes (˜1.5 mm) and numbers of SC-β cell clusters to achieve long-term euglycemia, likely due to lack of bioactive cues that support SC-β cell survival and function. Moreover, barium which was used for the alginate microsphere formation is toxic to humans, although it may elicit less immune response compared with other divalent cations.

The foregoing is illustrative of the present invention, and is not to be construed as limiting thereof. The invention is defined by the following claims, with equivalents of the claims to be included therein.

1. A composition comprising at least one in vitro-developed β-cell and a CXCL12 polypeptide encapsulated in a microcapsule.

2. The composition of claim 1, wherein the in vitro-developed β-cell is developed from pluripotent stem cells (SC-β cells).

3. The composition of claim 1, wherein the in vitro-developed β-cell is a human cell.

4. The composition of claim 1, wherein the CXCL12 polypeptide is incorporated in an amount of about 0.2 μg/ml to about 2.0 μg/ml.

5. The composition of claim 1, wherein the microcapsule is an alginate microcapsule.

6. The composition of claim 1, wherein the microcapsules have a diameter in the range of about 600 μm to about 700 μm.

7. A method of treating diabetes in a subject in need thereof, comprising delivering to the subject an effective amount of the composition of claim 1.

8. A method of accelerating the normalization of hyperglycemia in a subject in need thereof, comprising delivering to the subject an effective amount of the composition of claim 1, thereby accelerating the normalization of hyperglycemia.

9. A method of preventing fibrotic pericapsular overgrowth of microcapsules in a subject, comprising delivering to the subject an effective amount of the composition of claim 1, thereby preventing fibrotic pericapsular overgrowth of the microcapsules.

10. A method for enhancing a response against diabetes in a subject in need thereof, comprising delivering to the subject an effective amount of the composition of claim 1, thereby enhancing the response against diabetes.

11. The method of claim 7, wherein the CXCL12 polypeptide enhances the activity of the SC-β cells.

12. The method of claim 11, wherein the activity is increasing functional maturation.

13. The method of claim 11, wherein the activity is improving glucose-stimulated insulin secretion.

14. The method of claim 11, wherein the activity is improving survival.

15. The method of claim 11, wherein the activity is decreasing apoptosis.

16. The method of claim 11, wherein the activity is improving immunoisolation.
